(19) United States
(12) Patent Application Publication
(10) Pub. No.: US 2017/0041576 A1
(43) Pub. Date: Feb. 9, 2017

(54) MEDICAL IMAGING DEVICE, MEDICAL IMAGE ACQUISITION SYSTEM, AND ENDOSCOPE DEVICE

(71) Applicant: SONY OLYMPUS MEDICAL SOLUTINS INC., Tokyo (JP)

(72) Inventor: Motoaki KOBAYASHI, Tokyo (JP)

(73) Assignee: SONY OLYMPUS MEDICAL SOLUTINS INC., Tokyo (JP)

(21) Appl. No.: 15/211,549

(22) Filed: Jul. 15, 2016

(30) Foreign Application Priority Data
Aug. 4, 2015 (JP) 2015-154491
Jan. 29, 2016 (JP) 2016-016429

(51) Int. Cl. H04N 9/097; A61B 1/00; A61B 1/045; G06T 3/40

(52) U.S. Cl. CPC H04N 9/097; G06T 3/4015; A61B 1/00186; A61B 1/045; A61B 1/00045

(57) ABSTRACT

A medical imaging device includes: a spectroscopic unit configured to spectrally separate light from an outside into first light of a first color as a specific single color, and second light including specific second colors that are different from the first color; a first imaging element configured to include first pixels receiving the first light spectrally separated by the spectroscopic unit and converting the first light into an electrical signal; and a second imaging element configured to include: second pixels arranged with same array and interval as the first pixels of the first imaging element; and a color filter including filters each transmitting one of the second colors, the filters being arranged in accordance with the arrangement of the second pixels, the second imaging element being configured to receive the second light transmitted through the color filter and to convert the second light into an electrical signal.

### CROSS-REFERENCE TO RELATED APPLICATION(S)

The present application claims priority to and incorporates by reference the entire contents of Japanese Patent Application No. 2015-154491 filed in Japan on Aug. 4, 2015, and Japanese Patent Application No. 2016-016429 filed in Japan on Jan. 29, 2016.

### BACKGROUND

The present disclosure relates to a medical imaging device, a medical image acquisition system and an endoscope device.

In related art, there has been a demand for an increase in definition of a captured image in a medical image acquisition system, which captures a subject using an imaging element and observes the subject, in order to obtain a more elaborate observation image. An imaging device that receives observation light using a plurality of imaging elements, for example, in order to increase the definition is known (for example, see Japanese Laid-open Patent Publication No. 2007-235877). In the imaging device disclosed in Japanese Laid-open Patent Publication No. 2007-235877, a color separation prism that includes a plurality of prisms each of which is provided with a dichroic film to reflect or transmit light having wavelength bands different from each other and spectrally separate the observation light into light having four wavelength bands using the respective dichroic films, and four imaging elements that receive and image the observation light having the respective wavelength bands that have been spectrally separated by the color separation prism are provided. The imaging device realizes the high definition by setting pixels of one imaging element as a reference, and arranging positions of pixels of the other three imaging elements to be relatively shifted from the reference to increase the number of pixels.

In addition, an imaging device, which uses two imaging elements including an imaging element for acquisition of a brightness signal and an imaging element for generation of an observation image to receive light having wavelength bands of respective color components of red (R), green (G) and blue (B), is known as an imaging device that receives observation light using a plurality of imaging elements (for example, see JP 2010-130321 A). In the imaging device disclosed in JP 2010-130321 A, pixels of the imaging element for generation of the observation image are set as a reference, and positions of pixels of the imaging element for acquisition of the brightness signal are arranged to be relatively shifted from the reference. Further, the increase in definition is implemented by performing a demosaicing process to increase the number of pixels in a pseudo-manner using a signal value (pixel value) depending on the amount of light received by the pixels corresponding to the respective color components of RGB of the imaging element for generation of the observation image and the pixels of the imaging elements of the brightness signal.

### SUMMARY

Meanwhile, there is a demand for a so-called increase in quality of a captured image, including an increase in sensitivity of imaging sensitivity of an imaging unit, color reproducibility and the like, in addition to the increase in definition, in the medical image acquisition system. For example, the medical image acquisition system is generally used in combination with a light source device, which consistently illuminates a subject during observation, in order to obtain a bright observation image. There is the demand for the increase in sensitivity of imaging sensitivity of the imaging unit in the medical image acquisition system in order to obtain the bright observation image while implementing reduction in size and power saving of the light source device and suppression of heat generation. In addition, the color reproducibility is an important factor which is demanded in the medical image acquisition system.

In addition, there is a demand for reduction in size and weight of the imaging unit in the medical image acquisition system. For example, when an endoscope system which includes an imaging unit at a distal end of an insertion unit, there is the demand for the reduction in size and weight of the imaging unit in order for reduction in diameter and weight of the insertion unit. When an endoscope system includes an imaging unit in a camera head provided on a proximal end side of an optical scope such as a rigid scope, for example, there is the demand for the reduction in size and weight of the imaging unit in terms of reduction in size and weight of the camera head to allow an operator to grip and hold the camera head. In addition, when an operating microscope system includes an imaging unit in a microscope unit held by a support member such as an arm, for example, there is the demand for the reduction in size and weight of the imaging unit in order to increase a size and weight of the support member that supports the microscope unit.

However, it is necessary to use the color separation prism that spectrally separates the observation light into four in order to make the observation light incident to the respective imaging elements in the imaging device disclosed in JP 2007-235877 A, and accordingly, there is a problem that the size and weight of the device are increased due to the arrangement of the color separation prism. In addition, the observation light repeats a plurality of times of transmission and reflection with respect to the plurality of prism surfaces, including the dichroic films, after being transmitted through an initial incidence surface of the color separation prism until being transmitted through a final exit surface of the color separation prism in order to allow the observation light having the four different wavelength bands to be incident, respectively, to the four imaging elements. There is a risk that the observation light is attenuated through the plurality of times of transmission and reflection, the imaging sensitivity deteriorates, and the definition of the captured image decreases.

In addition, the imaging device disclosed in JP 2010-130321 A, the pixel of the imaging element for acquisition of the brightness signal (hereinafter, referred to as a Y pixel) has a spectral sensitivity of the same degree as a green wavelength band that is received by a G-component pixel of the imaging element for generation of the observation image (hereinafter, referred to as a G pixel). That is, a signal value of the wavelength band of the green component is obtained by not only the G pixel for green of the imaging element for generation of the observation image but also the Y pixel which is the entire pixel of the imaging element for acquisition of the brightness signal. On the other hand, signal values of wavelength bands of the red component and the blue component are obtained only by the pixels of the respective color components for red and blue of the single imaging element for generation of the observation image (hereinafter, the R-component pixel will be referred to as a R pixel and the B-component pixel will be referred to as a B pixel). Although the image with high definition in a pseudo-manner is obtained by using the two imaging elements, the number of the G pixels is extremely larger than the number of the R pixels and the B pixels, and accordingly, there is a risk that the color reproducibility of the image generated by the demosaicing process deteriorates. Not only the signal value of the wavelength band of the green component but also the signal values of the wavelength bands of the red component and the blue component are also subjected to the demosaicing process using the signal value of the wavelength band of the green component of the Y pixel Thus, there is the risk that the color reproducibility of the generated image deteriorates and the definition of the captured image is decreased.

The present disclosure has been made in view of the above description, and an object thereof is to provide a medical imaging device, a medical image acquisition system, and an endoscope device which are capable of suppressing an increase in size and weight thereof, and acquiring a high-quality observation image.

A medical imaging device according to one aspect of the preset disclosure may include: a spectroscopic unit configured to spectrally separate light from an outside into first light of a first color as a specific single color, and second light including specific second colors that are different from the first color; a first imaging element configured to include first pixels, the first pixels receiving the first light spectrally separated by the spectroscopic unit and converting the first light into an electrical signal; and a second imaging element configured to include: second pixels arranged with same array and interval as the first pixels of the first imaging element; and a color filter including filters each transmitting one of the second colors, the filters being arranged in accordance with the arrangement of the second pixels, the second imaging element being configured to receive the second light transmitted through the color filter and to convert the second light into an electrical signal, wherein the first and second imaging elements are arranged such that a pixel array of the second imaging element is shifted, with respect to a pixel array of the first imaging element, by a ½ pixel in at least one of two directions along an array direction when optical axes of the first and second light spectrally separated by the spectroscopic unit are set to match each other.

### BRIEF DESCRIPTION OF THE DRAWINGS

FIG. 1 is a diagram illustrating a schematic configuration of an endoscope device according to a first embodiment of the present disclosure;

FIG. 2 is a block diagram illustrating a configuration of a camera head and a control device illustrated in FIG. 1;

FIG. 3 is a schematic view for describing a configuration of an imaging unit according to the first embodiment of the present disclosure;

FIG. 4 is a schematic view illustrating a configuration of a pixel of an imaging element of the imaging unit according to the first embodiment of the present disclosure;

FIG. 5 is a schematic view for describing a configuration of a color filter of the imaging unit according to the first embodiment of the present disclosure;

FIG. 6 is a schematic view for describing the configuration of the color filter of the imaging unit according to the first embodiment of the present disclosure;

FIG. 7 is a schematic view for describing an arrangement of light (spots) acquired by the two imaging elements of the imaging unit according to the first embodiment of the present disclosure;

FIG. 8 is a schematic view for describing a configuration of an imaging unit according to a modified example of the first embodiment of the present disclosure;

FIG. 9 is a block diagram illustrating a configuration of a camera head and a control device of an endoscope device according to a second embodiment of the present disclosure;

FIG. 10 is diagram illustrating a schematic configuration of an endoscope device according to a third embodiment of the present disclosure;

FIG. 11 is a schematic view for describing a configuration of an imaging unit according to the third embodiment of the present disclosure;

FIG. 12 is a diagram illustrating a schematic configuration of an endoscope device according to a fourth embodiment of the present disclosure;

FIG. 13 is a diagram illustrating a configuration of the main section of the endoscope device according to the fourth embodiment of the present disclosure; and

FIG. 14 is a diagram schematically illustrating the overall configuration of an operating microscope system which is an observation system for medical use provided with a medical imaging device according to a fifth embodiment of the present disclosure.

### DETAILED DESCRIPTION

Hereinafter, a description will be given regarding modes for embodying the present disclosure (hereinafter, referred to as “embodiments”). A description will be given in the embodiments regarding an endoscope device for medical use that displays an in-vivo image of a subject, such as a patient, as an example of a medical image acquisition system which includes an imaging device according to the present disclosure. The disclosure is not limited by the embodiments. Further, the same reference sign will be assigned to the same components in the description of the drawings.

### First Embodiment

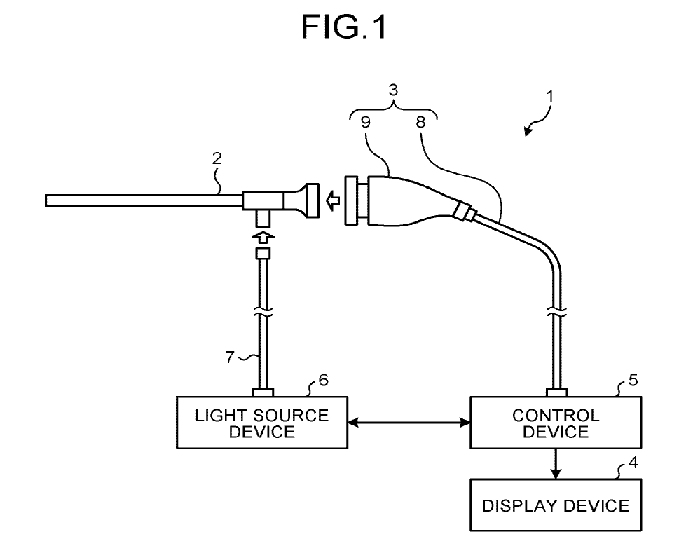

FIG. 1 is a diagram illustrating a schematic configuration of an endoscope device 1 according to a first embodiment of the present disclosure. The endoscope device 1 is a device which is used in the medical field and observes a (e.g., in-vivo) subject inside an object to be observed such as human. As illustrated in FIG. 1, this endoscope device 1 is provided with an endoscope 2, an imaging device 3 (e.g., a medical imaging device), a display device 4, a control device 5 (e.g., an image processing device), and a light source device 6, and a medical image acquisition system is configured to include the imaging device 3 and the control device 5. Incidentally, in the first embodiment, the endoscope device using a rigid scope is configured to include the endoscope 2 and the imaging device 3.

The light source device 6 has a light guide 7 of which one end is connected with the endoscope 2, and supplies white illumination light to the one end of the light guide 7 to illuminate the inside of a living body. The one end of the light guide 7 is detachably connected with the light source device 6, and the other end thereof is detachably to the endoscope 2. Further, the light guide 7 transmits the light supplied from the light source device 6 from the one end to the other end, and supplies the light to the endoscope 2.

The imaging device 3 captures a subject image from the endoscope 2, and outputs the imaging result. As illustrated in FIG. 1, the imaging device 3 is provided with a transmission cable 8, which is a signal transmission unit, and a camera head 9. In the first embodiment, a medical imaging device is configured to include the transmission cable 8 and the camera head 9.

The endoscope 2, which is rigid and has an elongated shape, is inserted into the living body. An optical system, which is configured to include one or a plurality of lenses and condenses the subject image, is provided inside the endoscope 2. The endoscope 2 emits the light supplied via the light guide 7 from a distal end thereof, and irradiates the inside of the living body with the emitted light. Then, the light with which the inside of the living body is irradiated (e.g., the subject image) is condensed by the optical system (e.g., a lens unit 91) inside the endoscope 2.

The camera head 9 is detachably connected with a proximal end of the endoscope 2. Further, the camera head 9 captures the subject image condensed by the endoscope 2 and outputs an imaging signal generated by the imaging, under the control of the control device 5. Incidentally, a detailed configuration of the camera head 9 will be described later.

The transmission cable 8 has one end which is detachably connected with the control device 5 via a connector, and the other end which is detachably connected with the camera head 9 via the connector. To be specific, the transmission cable 8 is a cable which includes a plurality of electrical wirings (not illustrated) arranged inside an outer cover serving as the outermost layer. The plurality of electrical wirings is electrical wirings which are configured to transmit the imaging signal output from the camera head 9, a control signal output from the control device 5, a synchronization signal, a clock, and power to the camera head 9.

The display device 4 displays an image generated by the control device 5 under the control of the control device 5. The display device 4 preferably includes a display unit which is equal to or larger than 55 inches, in order to easily obtain the sense of immersion during the observation, but is not limited thereto.

The control device 5 processes the imaging signal input from the camera head 9 via the transmission cable 8, outputs the imaging signal to the display device 4, and collectively controls the operation of the camera head 9 and the display device 4. Incidentally, a detailed configuration of the control device 5 will be described later.

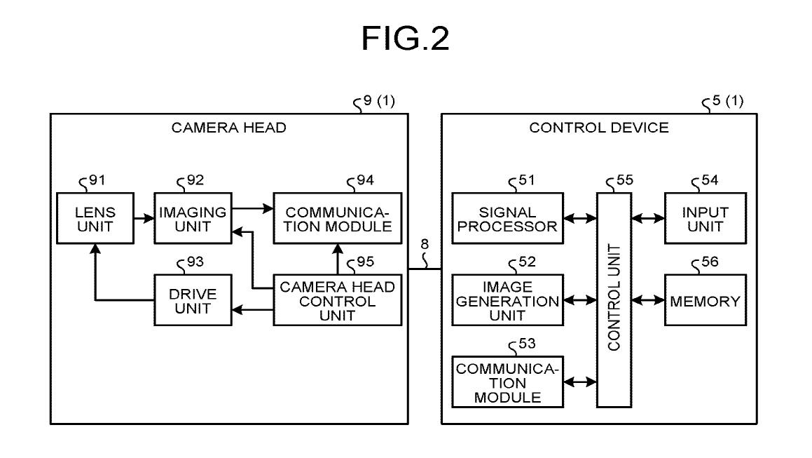

Next, a description will be given regarding each configuration of the imaging device 3 and the control device 5. FIG. 2 is a block diagram illustrating the configurations of the camera head 9 and the control device 5. Incidentally, FIG. 2 does not illustrate the connector which makes the camera head 9 and the transmission cable 8 to be attached to and detached from each other.

Hereinafter, the configuration of the control device 5 and the configuration of the camera head 9 will be described in the order. Incidentally, the main section of the present disclosure will be mainly described as the configuration of the control device 5, in the following description. As illustrated in FIG. 2, the control device 5 is provided with a signal processor 51, an image generation unit 52, a communication module 53, an input unit 54, a control unit 55, and a memory 56. Incidentally, the control device 5 may be provided with a power supply unit (not illustrated) and the like, which generates a power-supply voltage to drive the control device 5 and the camera head 9, supplies the voltage to each unit of the control device 5, and supplies the voltage to the camera head 9 via the transmission cable 8.

The signal processor 51 performs noise removal, or signal processing such as A/D conversion, if necessary, with respect to the imaging signal output from the camera head 9, thereby outputting the digitized imaging signal (e.g., a pulse signal) to the image generation unit 52.

In addition, the signal processor 51 generates the synchronization signal and the clock of the imaging device 3 and the control device 5. The synchronization signal (e.g., a synchronization signal to instruct an imaging timing of the camera head 9, and the like) and the clock (e.g., a clock for serial communication) with respect to the imaging device 3 are sent to the imaging device 3 via a line (not illustrated). The imaging device 3 is then driven based on the synchronization signal and the clock.

The image generation unit 52 generates an image signal for display, which is displayed by the display device 4, based on the imaging signal input from the signal processor 51. The image generation unit 52 generates the image signal for display, including the subject image, by executing a predetermined signal processing with respect to the imaging signal. Herein, examples of the image processing include various types of image processing such as interpolation processing, color correction processing, color enhancement processing, and contour enhancement processing. The image generation unit 52 outputs the generated image signal to the display device 4.

The communication module 53 outputs a signal, which includes a control signal to be described later that is transmitted from the control unit 55, from the control device 5, to the imaging device 3. In addition, the communication module 53 outputs a signal from the imaging device 3 to the control device 5. That is, the communication module 53 is a relay device that collects signals from the respective units of the control device 5, which are output to the imaging device 3, by, for example, parallel-to-serial conversion or the like, and outputs the collected signal, and that divides the signal input from the imaging device 3 by, for example, serial-to-parallel conversion or the like, and outputs the divided signals to the respective units of the control device 5.

The input unit 54 is implemented using a user interface such as a keyboard, a mouse, and a touch panel, and receives the input of various types of information.

The control unit 55 performs drive control of the respective components including the control device 5 and the camera head 9, and input and output control of the information with respect to the respective components. The control unit 55 generates the control signal by referring to communication information data (e.g., format information for communication and the like), which is recorded in the memory 56, and transmits the generated control signal to the imaging device 3 via the communication module 53. In addition, the control unit 55 outputs the control signal to the camera head 9 via the transmission cable 8.

The memory 56 is implemented using a semiconductor memory such as a flash memory and a dynamic random access memory (DRAM). In the memory 56, the communication information data (e.g., the format information for communication and the like) is recorded. Incidentally, various types of programs to be executed by the control unit 55 and the like may be recorded in the memory 56.

Incidentally, the signal processor 51 may include an AF processor, which outputs a predetermined AF evaluation value of each frame based the input imaging signals of the frames, and an AF calculation unit which performs AF calculation processing to select a frame, a focus lens position or the like that is the most suitable as a focusing position, from the AF evaluation values of the respective frames output from the AF processor.

Incidentally, the signal processor 51, the image generation unit 52, the communication module 53, and the control unit 55, described above, are implemented using a general-purpose processor such as a central processing unit (CPU) including an internal memory (not illustrated) in which the program is recorded, or dedicated processors including various types of arithmetic circuits and the like which execute specific functions such as an application specific integrated circuit (ASIC). In addition, they may be configured to include a field programmable gate array (FPGA) (not illustrated) which is one type of a programmable integrated circuit. Incidentally, in the case of being configured to include the FPGA, the FPGA, which is the programmable integrated circuit, may be configured by providing a memory to store configuration data, and using the configuration data read out from the memory

Next, the main section of the present disclosure will be mainly described as the configuration of the camera head 9. As illustrated in FIG. 2, the camera head 9 is provided with a lens unit 91, an imaging unit 92, a drive unit 93, a communication module 94, and a camera head control unit 95.

The lens unit 91 is configured to include one or a plurality of lenses, and forms the subject image condensed by the endoscope 2 on an imaging surface of the imaging element configuring the imaging unit 92. The one or plurality of lenses is configured to be movable along the optical axis. Further, the lens unit 91 is provided with an optical zoom mechanism (not illustrated) that changes an angle of view by moving the one or the plurality of lenses, and a focus mechanism that changes a focal point. Incidentally, the lens unit 91 may be provided with a diaphragm mechanism or an optical filter (e.g., a filter that cuts the infrared light) which is freely inserted and removed on the optical axis, in addition to the optical zoom mechanism and the focus mechanism.

The imaging unit 92 images the subject under the control of the camera head control unit 95. The imaging unit 92 is configured to include two imaging elements such as a charge coupled device (CCD) or a complementary metal oxide semiconductor (CMOS), which optically receives the subject image formed by the lens unit 91 and converts the received subject image into an electrical signal, and a prism which spectrally separates the observation light and makes the spectrally separated light incident to the two imaging elements, respectively. For example, in the case of the CCD, a signal processor (not illustrated), which performs signal processing (e.g., A/D conversion or the like) with respect to an electrical signal (e.g., an analog signal) from the imaging element, is mounted to a sensor chip or the like. For example, in the case of the CMOS, a signal processor, which performs signal processing (e.g., A/D conversion or the like) with respect to an electrical signal (analog) obtained by converting light into the electrical signal to output the imaging signal, is included in the imaging element. A configuration of the imaging unit 92 will be described later.

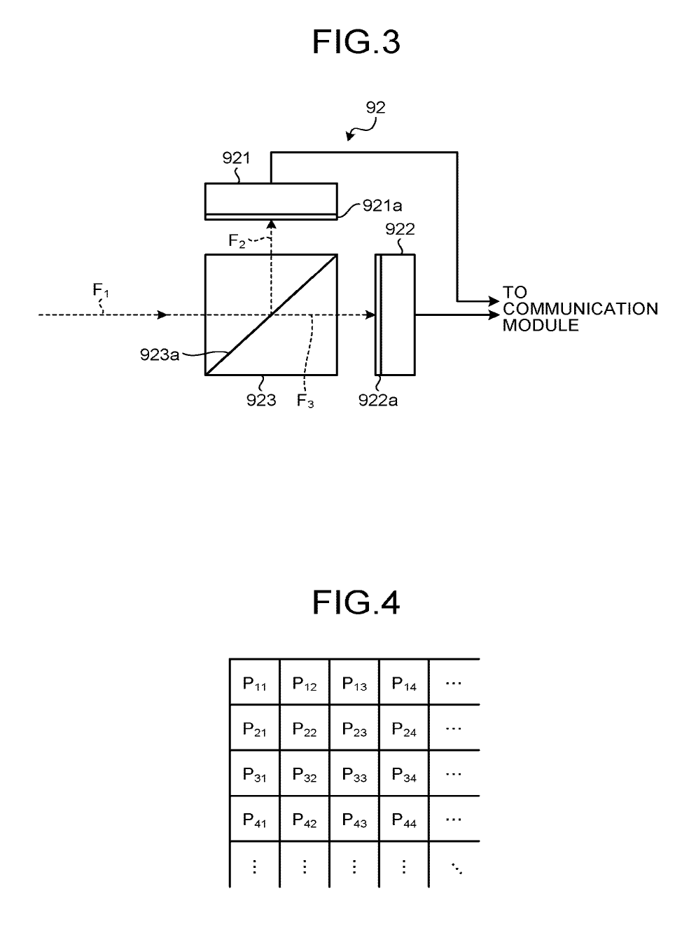

FIG. 3 is a schematic view for describing the configuration of the imaging unit 92. As illustrated in FIG. 3, the imaging unit 92 includes a first imaging element 921, a second imaging element 922, and a prism 923. The observation light from the outside is incident to the prism 923 via the lens unit 91, and the light spectrally separated by the prism 923 is incident to the first imaging element 921 and the second imaging element 922 in the imaging unit 92.

FIG. 4 is a schematic view illustrating a configuration of a pixel of the imaging element of the imaging unit 92. Hereinafter, although a pixel configuration of the first imaging element 921 has been described with reference to FIG. 4, the second imaging element 922 has the same configuration. A pixel array of the second imaging element 922 has the same array and interval as those of a pixel array of the first imaging element 921. The first imaging element 921 has a plurality of the pixels which receives the light from the lens unit 91 and the prism 923 and are two-dimensionally arranged like a square (e.g., arranged in a matrix form). Further, the first imaging element 921 generates an electrical signal (also known as an imaging signal or the like) by performing photoelectric conversion with respect to the light received by the respective pixels. This electrical signal includes each pixel value (e.g., a brightness value) of the pixels and position information of the pixels. In FIG. 4, a pixel arranged at a row x and a column y is denoted by a pixel P_{xy} (e.g., x and y are natural numbers).

The first imaging element 921 and the second imaging element 922 are arranged such that each light receiving surface of the pixels receiving light is arranged at a conjugate position with respect to a focal plane of the lens unit 91, and a position of a pixel P_{xy} of the first imaging element 921 and a position of a pixel P_{xy} of the second imaging element 922 are shifted by a ½ pixel in each of the row direction and the column direction serving as the array direction of the pixel array with respect to the optical axis of the observation light. For example, when the first imaging element 921 and the second imaging element 922 are superimposed on each other by aligning each optical axis of the observation light, a position of a pixel P₁₁ of the second imaging element 922 is shifted by a ½ pixel in each of the row direction and the column direction of the array direction of the pixel array of the first imaging element 921 with respect to a position of a pixel P₁₁ of the first imaging element 921. The first imaging element 921 and the second imaging element 922 are fixed by a fixing member (not illustrated) in a state in which the optical-axis direction of the observation light, the yaw direction, the roll direction, the pitch direction, and two axial directions (e.g., the horizontal direction and the perpendicular direction) which are orthogonal to each other in a plane perpendicular to the optical axis are adjusted.

In addition, the first imaging element 921 and the second imaging element 922 include color filters 921a and 922a, respectively, which are provided on the light receiving surface. The color filters 921a and 922a include a plurality of filters that transmits the light having the respective wavelength bands that are individually set. The color filters 921a and 922a may be provided to be bonded to each light receiving surface of the first imaging element 921 and the second imaging element 922, or may be provided to be integrated with each of the first imaging element 921 and the second imaging element 922. In addition, the color filters 921a and 922a may be provided with the plurality of filters in an integrated manner, or may include the individual filters being provided in the respective light receiving surfaces.

The color filters 921a and 922a are obtained by arranging the filters, which transmit predetermined light (e.g., a first color or a second color), side by side in a matrix form according to the arrangement of the pixels P_{xy}. In other words, each one of the filters, which transmit the light having the predetermined wavelength bands, is arranged in each light receiving surface of the pixels. Thus, the pixel P_{xy} provided with a filter receives light having a wavelength band that is transmitted through the filter. For example, the pixel P_{xy}, which is provided with a filter that transmits light having a wavelength band of green (G) receives light having a wavelength band of green. Hereinafter, the pixel P_{xy} that receives the light having the wavelength band of green will be referred to as a G pixel. In the same manner, a pixel that receives light having a wavelength band of red (R) will be referred to as an R pixel, and a pixel that receives light having a wavelength band of blue (B) will be referred to as a B pixel. Herein, each wavelength band of the blue, green and red is set such that, for example, the wavelength band of blue is 380 nm to 500 nm, the wavelength band of green is 480 nm to 600 nm, and the wavelength band of red is 580 nm to 650 nm. Hereinafter, a description will be given regarding a case in which the green is set as the first color, and the blue and red are set as the second color in the first embodiment.

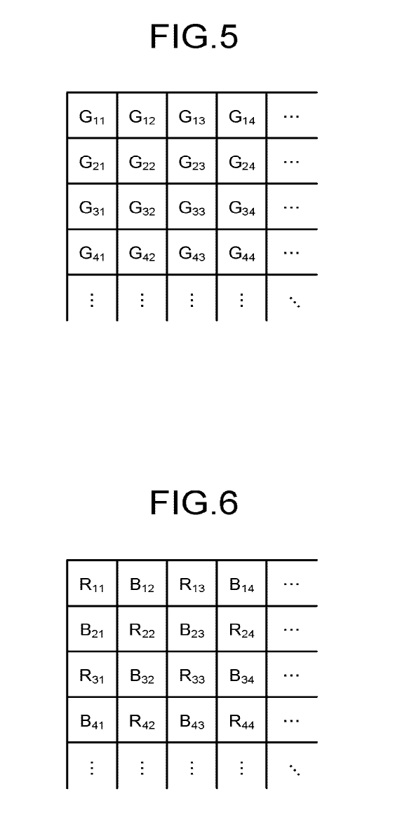

FIG. 5 is a schematic view for describing a configuration of the color filter of the imaging unit according to the first embodiment of the present disclosure, and is the schematic view illustrating an example of a configuration of the color filter 921a provided in the first imaging element 921. The color filter 921a includes filters which transmit the light having the wavelength band of green (G filters) and are arranged side by side in a matrix form according to the arrangement of the pixels P_{xy}. Thus, the respective pixels P_{xy} receive the light having the wavelength band of green and transmitted through the color filter 921a in the first imaging element 921.

FIG. 6 is a schematic view for describing a configuration of the color filter of the imaging unit according to the first embodiment of the present disclosure, and is the schematic view illustrating an example of a configuration of the color filter 922a provided in the second imaging element 922. The color filter 922a includes filters which transmit the light having the wavelength band of red (R filters) and filters which transmit the light having the wavelength band of blue (B filters), both of which are arranged side by side in a matrix form according to the arrangement of the pixels P_{xy}. The R filter and the B filter are alternately arranged along the row direction and the column direction. Thus, the respective pixels receive either of the light having the wavelength band of red and the light having the wavelength band of blue which have passed through the color filter 922a in the second imaging element 922.

The prism 923 has a cubic shape obtained by bonding two prisms each of which has a triangular prism shape, and a dichroic film 923a, which is a thin film, is provided between bonding surfaces. The dichroic film 923a reflects the first light having the wavelength band of green, and passes the second light having the wavelength bands of blue and red. Thus, observation light F₁ incident to the prism 923 is spectrally separated into first light F₂ having the wavelength band of green and second light F₃ having the wavelength bands of red and blue. The respective spectrally separated light (the first light F₂ and the second light F₃) are emitted from different outer surfaces (planes) of the prism 923 to the outside (see FIG. 3). The prism 923 spectrally separates the observation light F₁ into the first light F₂ and the second light F₃ through one time of reflection and transmission. In the first embodiment, the first light F₂ having the wavelength band of green is incident to the first imaging element 921 (e.g., to the color filter 921a), and the second light F₃ having the wavelength bands of red and blue is incident to the second imaging element 922 (e.g., to the color filter 922a). Incidentally, the prism 923 may have a rectangular parallelepiped shape or a polygonal shape other than the cubic shape, as long as the incident light may be spectrally separated and the spectrally separated light may be emitted.

Meanwhile, human eyes perceive the light of green to be bright due to the luminosity characteristic. In the embodiment, it is configured such that the first imaging element 921 outputs an imaging signal of green and the second imaging element 922 outputs imaging signals of red and blue in order to acquire an image makes human feel bright while securing the color balance (color reproducibility) among the red (R), the green (G) and the blue (B).

The drive unit 93 includes a driver which operates the optical zoom mechanism or the focus mechanism, and changes the angle of view or the position of the focal point of the lens unit 91, under the control of the camera head control unit 95.

The communication module 94 outputs the signal transmitted from the control device 5 to the respective units inside the camera head 9 such as the camera head control unit 95. In addition, the communication module 94 converts the information relating to a current state of the camera head 9 into a signal format according to a transmission scheme which has been set in advance, and outputs the converted signal to the control device 5 via the transmission cable 8. That is, the communication module 94 is a relay device that divides the signal input from the control device 5 and the transmission cable 8 by, for example, the serial-to-parallel conversion or the like and outputs the divided signals to the respective units of the camera head 9, and that collects signals from the respective units of the camera head 9 output to the control device 5 and the transmission cable 8 by, for example, the parallel-to-serial conversion or the like and outputs the collected signal.

The camera head control unit 95 controls the operation of the entire camera head 9 according to a drive signal input via the transmission cable 8 and an instruction signal output from an operation unit when the user operates the operation unit, such as a switch, which is provided to be exposed on an external surface of the camera head 9. In addition, the camera head control unit 95 outputs the information relating to the current state of the camera head 9 to the control device 5 via the transmission cable 8.

Incidentally, the drive unit 93, the communication module 94, and the camera head control unit 95, described above, are implemented using the general-purpose processor such as the central processing unit (CPU) including the internal memory (not illustrated) in which the program is recorded, or the dedicated processors including various types of arithmetic circuits which execute specific functions such as the application specific integrated circuit (ASIC). In addition, they may be configured to include the FPGA which is one type of the programmable integrated circuit. Incidentally, in the case of being configured to include the FPGA, the FPGA, which is the programmable integrated circuit, may be configured by providing a memory to store configuration data, and using the configuration data read out from the memory.

Incidentally, the camera head 9 and the transmission cable 8 may be configured to include a signal processor which executes signal processing with respect to the imaging signal generated by the communication module 94 or the imaging unit 92. In addition, an imaging clock to drive the imaging unit 92 and a driving clock to drive the drive unit 93 may be generated based on a reference clock generated by an oscillator (not illustrated) provided inside the camera head 9, and be output to the imaging unit 92 and the drive unit 93, respectively. Alternatively, timing signals of various types of processing in the imaging unit 92, the drive unit 93, and the camera head control unit 95 may be generated based on the synchronization signal input from the control device 5 via the transmission cable 8, and be output to each of the imaging unit 92, the drive unit 93, and the camera head control unit 95. The camera head control unit 95 may be provided not in the camera head 9, but in the transmission cable 8 or the control device 5.

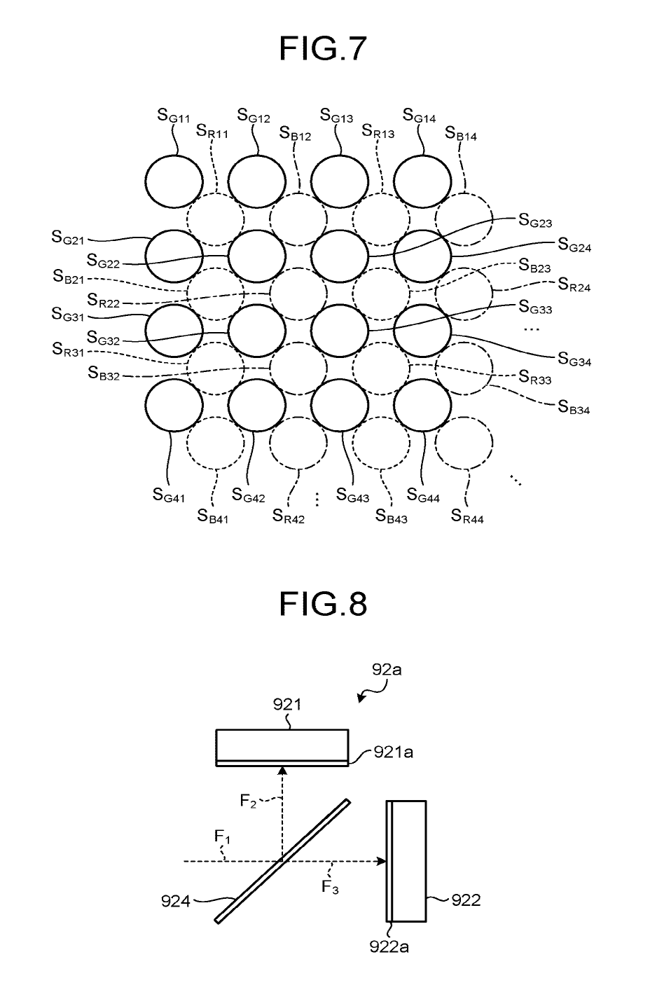

Next, a description will be given regarding the imaging signal to be obtained by the first imaging element 921 and the second imaging element 922, with reference to FIG. 7. FIG. 7 is a schematic view for describing an arrangement of light (spots) acquired by the two imaging elements of the imaging unit according to the first embodiment. Incidentally, FIG. 7 schematically illustrates the light incident to the respective pixels via the color filter using a circle (spot). For example, the light, which is incident to the pixel P₁₁ after being transmitted through a G₁₁ of the color filter 921a, is set as a spot S_{G11}; the light, which is incident to the pixel P₁₁ after being transmitted through a R₁₁ of the color filter 922a, is set as a spot S_{R11}; and the light, which is incident to the pixel P₁₂ after being transmitted through a B₁₂ of the color filter 922a, is set as a spot S_{B12}.

The spots received by the respective pixels of the first imaging element 921, that is, spots formed by the light transmitted through the G filter (for example, spots S_{G11} to S_{G44} illustrated in FIG. 7) are arranged in a matrix form. In addition, the spots received by the respective pixels of the second imaging element 922, that is, spots formed by the light transmitted through the R filter or the B filter (for example, spots S_{R11} to S_{R44} and S_{D12} to S_{D43} illustrated in FIG. 7) are spots in which color components of adjacent spots are different from one another, and are arranged in a matrix form.

When the arrangement of the respective spots received by the respective pixels of the first imaging element 921 and the arrangement of the respective spots received by the respective pixels of the second imaging element 922 are superimposed on each other by aligning each optical axis of the observation light, the positions of the pixels the first imaging element 921 and the second imaging element 922 are shifted from each other by the ½ pixel in each of the row direction and the column direction, and thus, the spots S_{R11} to S_{R44} and S_{D12} to S_{D43}, formed by the light transmitted through the R filter or the B filter are arranged among the spots S_{G11} to S_{G44} formed by the light transmitted through the G filter as illustrated in FIG. 7. In other words, a state is formed in which the spots S_{G11} to S_{G44}, formed by the light transmitted through the G filter, and the spot S_{R11} to S_{R44}, S_{R12} to S_{R43}, formed by the light transmitted through the R filter or the B filter, are arranged side by side to be independent from each other when seen in the row direction of the pixel P_{xy}, for example.

In this manner, when the pixel positions with respect to the optical axes of the first imaging element 921 and the second imaging element 922 are shifted by the ½ pixel in each of the row direction and the column direction, it is possible to make the number of spots double when seen in any one of the row direction and the column direction, as long as using the imaging elements having the same number of pixels. Thus, when the brightness values of the RGB components are given to all the spots by interpolating the color components for each of the spots, the number of pixels in any one of the row direction and the column direction becomes double among the number of pixels of the image signal for display generated by the image generation unit 52, and it is possible to regard that the definition thereof is doubled. Herein, it is possible to use a known method such as a nearest neighbor method, a bilinear method, and a bicubic method as the interpolation processing.

To be specific, when an imaging element having the number of pixels in response to an image signal of standard definition (SD) is used, the image signal may be considered to correspond to an image signal of high definition (HD). Further, when an imaging element having the number of pixels in response to an image signal of HD is used, the image signal may be considered to correspond to an image signal of 4K, which is higher definition. When an imaging element having the number of pixels in response to an image signal of 4K, the image signal may be considered to correspond to an image signal of 8 k, which is still higher definition. Herein, the image signal of SD is an image signal which has the definition approximately with 720 pixels in the row direction and 480 pixels in the column direction, for example. The image signal of HD is an image signal which has the definition approximately with 1920 pixels in the row direction and 1080 pixels in the column direction, for example. The image signal of 4K is an image signal which has the definition approximately with 3840 pixels in the row direction and 2160 pixels in the column direction, for example. The image signal of 8K is an image signal which has the definition approximately with 7680 pixels in the row direction and 4320 pixels in the column direction, for example.

According to the first embodiment described above, the prism 923 selectively spectrally separates the light incident from the outside using the wavelength band in response to the color component, and makes the spectrally separated light incident only to each of the two imaging elements of the first imaging element 921 and the second imaging element 922. Further, as the prism 923 arranges the pixel positions, with respect to the optical axes of the first imaging element 921 and the second imaging element 922, to be shifted by the ½ pixel in each of the row direction and the column direction, it is possible to acquire an observation image of high definition.

In addition, the spectral separation is performed through the one-time reflection and transmission using the prism 923, it is possible to provide the easier configuration and reduce the size as compared to a prism that emits light, which is spectrally separated as being folded a plurality of times, to the outside. As a result, it is possible to reduce the size and weight of the entire device according to the above-described first embodiment. In addition, the number of times of reflection and transmission of the observation light between the observation light is incident to the prism and emitted therefrom is minimally set to one, and thus, it is possible to suppress the attenuation of the observation light in an optical path to the first imaging element 921 and the second imaging element 922, and improve the imaging sensitivity.

In addition, the light having the wavelength band of green is spectrally separated from the observation light, and is incident to the first imaging element 921 of one side so that the first imaging element 921 outputs the imaging signal of green. Further, the light having the wavelength bands of blue and red is spectrally separated from the observation light, and is incident to the second imaging element 922 of the other side so that the second imaging element 922 outputs the imaging signal of blue and red according to the above-described first embodiment. Accordingly, it is possible to secure the color balance (color reproducibility) among the red (R), the green (G) and the blue (B), and to generate a high-quality image which is clear and bright in accordance with the luminosity characteristic.

Although the description has been given, in the above-described first embodiment, regarding a case in which the pixel positions, with respect to the optical axes of the first imaging element 921 and the second imaging element 922, are shifted by the ½ pixel in each of the row direction and the column direction, it may be enough as long as the pixel position is shifted in a direction to double the number of pixels in the image signal to be generated. That is, the pixel position with respect to the optical axes of the first imaging element 921 and the second imaging element 922 may be set such that the pixels are shifted in the direction to double the number of pixels in the image signal. The pixel position with respect to the optical axes of the first imaging element 921 and the second imaging element 922 may be set such that the pixels are shifted in at least any one of the two directions (the row direction and the horizontal direction) along the array direction of the pixel array.

In addition, the description has been given, in the above-described first embodiment, regarding a case in which the color filter of the first imaging element 921 is formed as the G filter, and the color filters of the second imaging element 922 are formed as the R filter and the B filter; however, it may be configured such that color filters of the first imaging element 921 are formed as the R filter and the B filter, and a color filter of the second imaging element 922 is formed as the G filter by setting each wavelength band of light that the prism 923 reflects and passes to be reversed from the respective wavelength bands described above, for example. Since the green (G) color component has a higher degree of visibility as compared to the red (R) color component and the blue (B) color component, it is preferable to arrange the filters of the respective colors such that the color filter of one imaging element is formed only as the G filter, and the color filters of the other imaging element is formed as the R filter and the B filter, in terms of generating an image with the high visibility. However, the disclosure is not limited thereto, and may be configured such that, for example, the color filter of the first imaging element 921 is formed as the B filter, the color filters of the second imaging element 922 are formed as the R filter and the G filter, and a wavelength band of light that the prism 923 reflects and passes is selected in accordance with a color of each filter of these imaging element. Combinations of the G filter, the R filter, and the B filter to be arranged in the two imaging elements may be arbitrarily set.

In addition, the description has been given, in the above-described first embodiment, regarding a case in which the first imaging element 921 receives light via the color filter 921a formed using the G filter; however, the first imaging element 921 may directly receive light from the prism 923 without the color filter 921a being provided, in the case of receiving light having a wavelength band of a single color.

### Modified Example of First Embodiment

Subsequently, a modified example of the first embodiment of the present disclosure will be described. FIG. 8 is a schematic view for describing a configuration of an imaging unit according to the modified example of the first embodiment of the present disclosure. Although the description has been given, in the above-described first embodiment, regarding the case of using the prism 923 to perform the spectral separation, the spectral separation is performed using a plate-shaped membrane mirror 924 which is provided with a dichroic film in this modified example.

An imaging unit 92a according to the modified example includes the first imaging element 921, the second imaging element 922, and the membrane mirror 924. In the imaging unit 92a, observation light F₁ from the outside is incident to the membrane mirror 924 via the lens unit 91, and light, which is spectrally separated by the membrane mirror 924, is incident to the first imaging element 921 and the second imaging element 922.

The membrane mirror 924 has a plate shape in which the dichroic film is provided on a surface on the lens unit 91 side. This dichroic film reflects the light having the wavelength band of green and passes the wavelength bands of red and blue similarly to the dichroic film 923a described above. Thus, the observation light F₁ incident to the membrane mirror 924 is spectrally separated into light F₂ having the wavelength band of green and light F₃ having the wavelength bands of red and blue.

According to the modified example, the spectral separation is performed using the plate-shaped membrane mirror 924, and thus, it is possible to obtain the effect of the above-described first embodiment, and further, to reduce the weight thereof as compared to the case of using the prism 923. Incidentally, the description has been given, in the above-described modified example, regarding the configuration in which the dichroic film is provided in the plate-shaped member serving as the membrane mirror 924; however, it may be configured to include a membrane mirror, for example, a pellicle mirror or the like having a thickness of equal to or smaller than 0.1 mm in order to further suppress the influence of degradation in optical performance caused depending on the thickness of the mirror of the membrane mirror 924.

### Second Embodiment

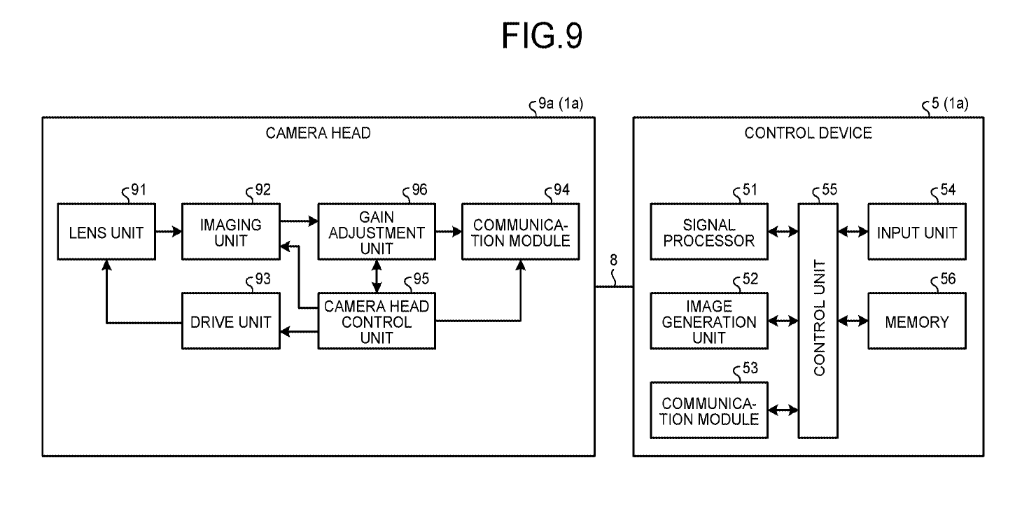

Next, a second embodiment of the present disclosure will be described. FIG. 9 is a block diagram illustrating a configuration of a camera head and a control device of an endoscope device according to a second embodiment of the present disclosure. Incidentally, a description will be given by assigning the same reference sign to the same configuration as the above-described configuration. Although the description has been given, in the above-described first embodiment, regarding a case in which the communication module 94 outputs the signal having been input from the imaging unit 92, to the control device 5, a signal generated by the imaging unit 92 is subjected to gain adjustment processing in the second embodiment.

An endoscope device 1a according to the second embodiment is provided with the endoscope 2, the imaging device 3, the display device 4, the control device 5, and the light source device 6 which are described above. In the second embodiment, the imaging device 3 includes a camera head 9a instead of the camera head 9.

The camera head 9a is provided with the lens unit 91, the imaging unit 92, the drive unit 93, the communication module 94, and the camera head control unit 95, which are described above, and a gain adjustment unit 96.

The gain adjustment unit 96 performs the gain adjustment processing with respect to an imaging signal, which is an electrical signal input from the imaging unit 92, and inputs the gain-adjusted imaging signal to the communication module 94. The gain adjustment unit 96 performs the gain adjustment processing by multiplying a signal generated by the first imaging element 921 and a signal generated by the second imaging element 922 by different gain coefficients, for example. To be specific, the gain adjustment unit 96 multiplies the signal of the G component generated by the first imaging element 921 by a gain coefficient α, and multiplies the signal of the R component and the B component generated by the second imaging element 922 by a gain coefficient β. The gain coefficients α and β may be arbitrarily set according to the luminosity characteristic and the color reproducibility, for example. Incidentally, the gain adjustment unit 96 may be provided not in the camera head 9 but in the transmission cable 8 or the control device 5.

According to the second embodiment, the same effect according to the first embodiment is obtained; and further, it is possible to perform the brightness adjustment for each spectrally separated signal since the gain adjustment unit 96 is configured to perform the gain adjustment processing with respect to the signals generated by the first and second imaging elements.

### Third Embodiment

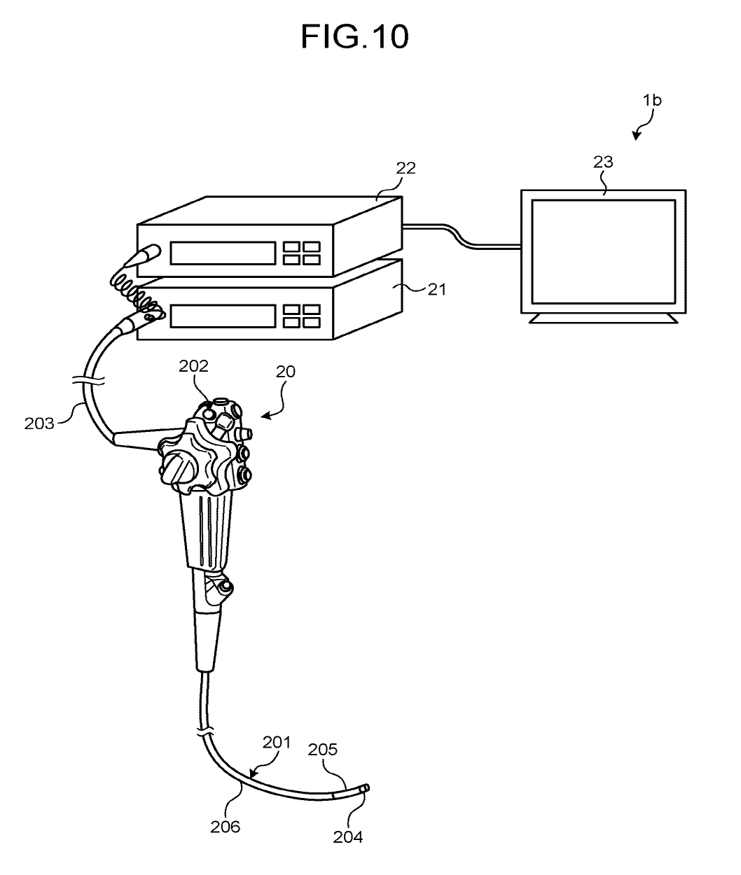

Subsequently, a third embodiment of the present disclosure will be described. FIG. 10 is a diagram illustrating a schematic configuration of an endoscope device 1b according to the third embodiment of the present disclosure. Although the description has been given, in the above-described first embodiment, regarding the endoscope device 1 in which the rigid scope is used as the endoscope 2, the endoscope device is not limited thereto. The endoscope device may use a flexible scope as the endoscope 2. In the third embodiment, a description will be given by exemplifying a case in which an imaging unit is provided at a distal end of an insertion unit of a flexible endoscope.

The endoscope device 1b is provided with an endoscope 20 which captures an in-vivo image of a part to be observed by inserting an insertion unit 201 inside a subject and generates an imaging signal, a light source device 21 which generates illumination light to be emit from a distal end of the endoscope 20, a control device 22 which performs predetermined imaging processing to the imaging signal acquired by the endoscope 20 and collectively controls the operation of the entire endoscope device 1b, and a display device 23 which displays the in-vivo image subjected to the image processing by the control device 22. The endoscope device 1b inserts the insertion unit 201 inside the subject such as a patient, and acquires the in-vivo image inside the subject. Incidentally, the control device 22 has functions of the signal processor 51, the image generation unit 52 and the like described above.

The endoscope 20 is provided with the insertion unit 201 which is flexible and has an elongated shape, an operation unit 202 which is connected with a proximal end side of the insertion unit 201 and receives input of various types of operation signals, and a universal code 203 which extends from the operation unit 202 in a direction different from an extending direction of the insertion unit 201 and include various types of cables, being built-in, to be connected with the light source device 21 and the control device 22.

The insertion unit 201 includes a distal end portion 204 including a built-in imaging unit 92b according to the third embodiment, a folding portion 205 which is configured to include a plurality of folding pieces and is freely folded, and a flexible tube portion 206 which is connected with a proximal end side of the folding portion 205, is flexible, and has an elongated shape.

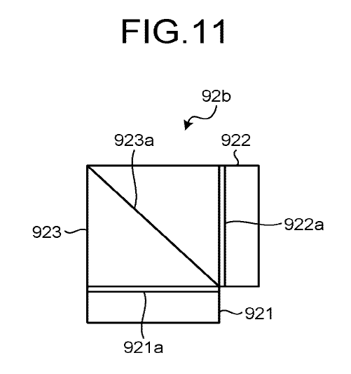

FIG. 11 is a schematic view for describing a configuration of the imaging unit according to the third embodiment of the present disclosure. The imaging unit 92b includes the first imaging element 921, the second imaging element 922, and the prism 923 similarly to the imaging unit 92. In the imaging unit 92b, the respective light receiving surfaces (the color filters 921a and 922a) of the first imaging element 921 and the second imaging element 922 are arranged, respectively, in different surfaces of the prism 923. The respective arrangement surfaces of the first imaging element 921 and the second imaging element 922 on the prism 923 are preferably surfaces which are orthogonal to each other.

In addition, when a thin film substrate such as an FPC board is used to electrically connect the first imaging element 921 and the second imaging element 922, with the communication module 94 and the like, it is possible to further reduce the thickness of the imaging unit 92b.

When the imaging unit 92b according to the third embodiment is used, it is possible to suppress an increase in diameter of the insertion unit 201 even in the case of being provided in the distal end portion 204 of the insertion unit 201 of the flexible endoscope.

### Fourth Embodiment

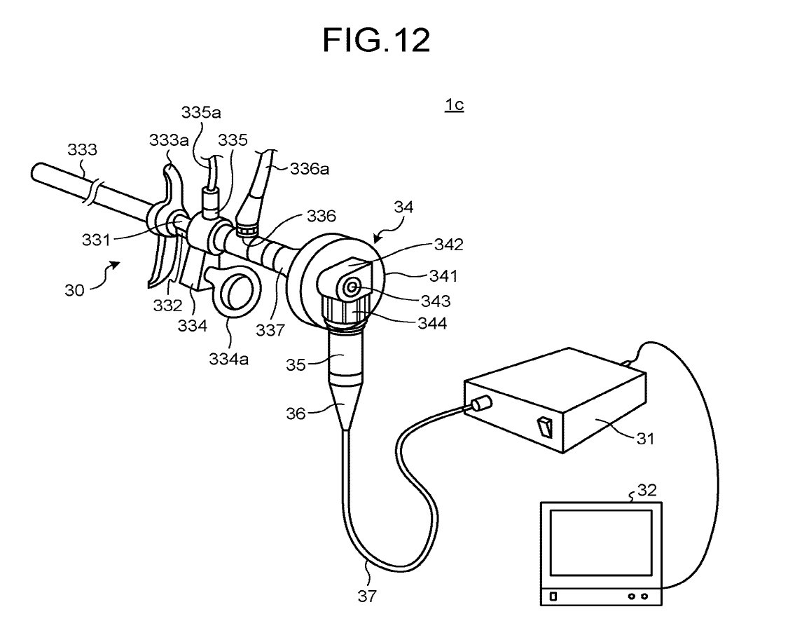

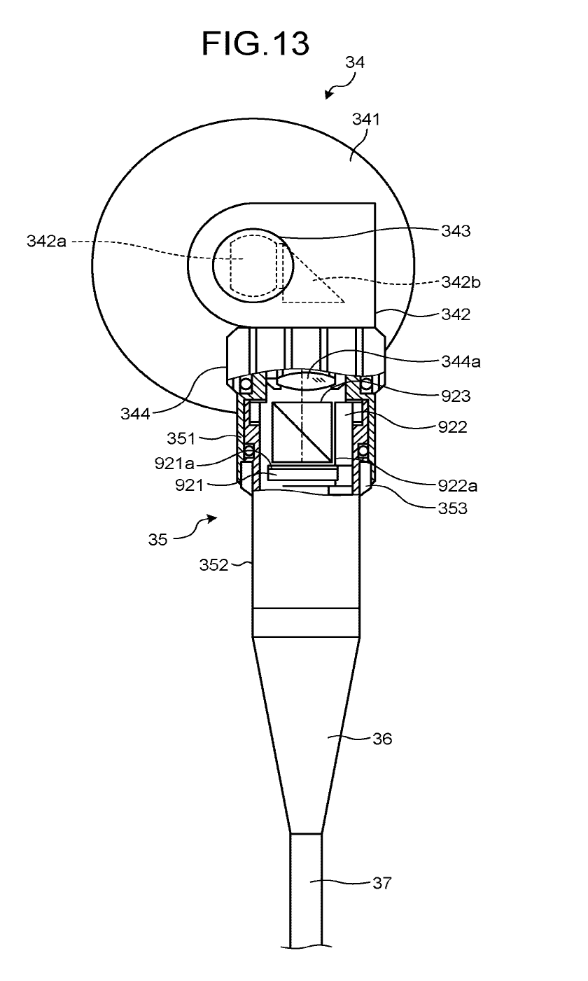

FIG. 12 is a diagram illustrating a schematic configuration of an endoscope device 1c according to a fourth embodiment of the present disclosure. FIG. 13 is a diagram illustrating a configuration of the main section of the endoscope device according to the fourth embodiment of the present disclosure. The endoscope device 1c is a device which is used in the medical field, and observes an inner part of an object to be observed such as human (e.g., the inside of a living body), in particular, an inner part of the ear or nose. As illustrated in FIG. 12, the endoscope device 1c is provided with an endoscope 30, a control device 31 (e.g., an image processing device), a display device 32, and an imaging device 34 (e.g., a medical imaging device), and a medical image acquisition system is configured to include the imaging device 34 and the control device 31. Incidentally, an endoscope device using a rigid scope is configured to include the endoscope 30 and the imaging device 34 in the fourth embodiment.

The endoscope 30, which is rigid and has an elongated shape, is inserted inside the living body. The endoscope 30 includes an optical scope 331, a resecto-electrode member 332, a sheath 333, a slide operation member 334, a power connector 335, a light source connector 336, and an eyepiece portion 337.

The optical scope 331 is provided with an optical system which is configured to include one or a plurality of lenses and condenses a subject image. The resecto-electrode member 332 may transmit a high-frequency current under the control of the control device 31, and it is possible to perform an incision of a living body tissue, using the high-frequency current. The optical scope 331 and the resecto-electrode member 332 are inserted inside the sheath 333, and are provided in parallel at a distal end of the sheath 333. In addition, a grip handle 333a is provided on a proximal end side of the sheath 333.

The slide operation member 334 holds the resecto-electrode member 332, and may manipulate a position of the resecto-electrode member 332 with respect to the sheath 333 by moving forward and backward. The slide operation member 334 is provided with a finger hooking handle 334a which is hooked by a finger of the user at the time of performing the forward and backward movement of the slide operation member 334.

The power connector 335 is provided in an upper end portion of the slide operation member 334, and is connected with a power cord 335a which is connected with a high-frequency power supply (not illustrated). The power connector 335 supplies the high-frequency current, which is supplied via the power cord 335a, to the resecto-electrode member 332. In addition, the light source connector 336 is provided in an upper end portion of the optical scope 331, and is connected with a light guide cable 336a which is connected with a light source (not illustrated). The light source connector 336 supplies illumination light, which is supplied via the light guide cable 336a, to the optical scope 331.

The endoscope 30 emits the light, which is supplied via the light guide cable 336a, from a distal end thereof, and irradiates the inside of the living body with the light. The light with which the inside of the living body is irradiated (e.g., a subject image) is condensed by the optical system inside the endoscope 30.

The imaging device 34 captures the subject image from the endoscope 2, and outputs the imaging result. As illustrated in FIG. 12, the imaging device 34 is provided with a camera head 35, a folding stopping tube 36, and a transmission cable 37 which is a signal transmission unit. In the fourth embodiment, a medical imaging device is configured to include the camera head 35 and the transmission cable 37.

The imaging device 34 is detachably connected with the eyepiece portion 337 of the endoscope 30. The imaging device 34 is provided with a disc-shaped mounting member 341 which is configured to be connected with the eyepiece portion 337, an imaging adapter 342 which is rotatable about an optical axis of observation light received by the optical scope 331, that is, a central axis of the optical scope 331, with respect to the mounting member 341, an eyepiece portion for macroscopic observation 343 with which an observation image from the optical scope 331 may be observed with the naked eye via the imaging adapter 342, and a cylindrical coupling portion 344 which is coupled with the camera head 35. A positioning of the imaging adapter 342 with respect to the mounting member 341 is implemented using a known method such as a click mechanism.

In addition, the imaging adapter 342 includes therein a beam splitter 342a which may fold a part of the observation light from the optical scope 331 and transmit the other observation light, and a triangular prism 342b to which the observation light being folded by the beam splitter 342a is incident and which folds the incident observation light toward the camera head 35. In addition, a focus adjusting optical system, which includes a lens 344a, is formed inside the coupling portion 344. The coupling portion 344 is rotatable around a communication axis, and may adjust a lens position (e.g., an image forming position) of the focus adjusting optical system by rotating.

The camera head 35 captures the subject image condensed by the endoscope 30 under the control of the control device 31, and outputs the imaging signal obtained by the imaging. The camera head 35 is provided with a cylindrical main body portion 351, a cylindrical lens barrel 352, and a fixing ring 353 that fixes the main body portion 351 and the lens barrel 352. In addition, the camera head 35 includes therein, for example, the first imaging element 921 provided with the color filter 921a, the second imaging element 922 provided with the color filter 922a, the prism 923, the communication module 94, and the camera head control unit 95.

One end of the transmission cable 37 is detachably connected with the control device 31 via a connector, and the other end thereof is detachably connected with the camera head 35 via a connector and the folding stopping tube 36. To be specific, the transmission cable 37 is a cable in which a plurality of electrical wirings (not illustrated) is arranged inside an outer cover serving as the outermost layer. The plurality of electrical wirings is electrical wirings which are configured to transmit the imaging signal output from the camera head 35, a control signal output from the control device 31, a synchronization signal, a clock, and power to the camera head 35.

The display device 32 displays an image generated by the control device 31 under the control of the control device 31.

The control device 31 processes the imaging signal, which is input from the camera head 35 via the transmission cable 37, outputs an image signal to the display device 32, and collectively controls each operation of the camera head 35 and the display device 32. Incidentally, the control device 31 has functions of the signal processor 51, the image generation unit 52 and the like described above.

In the endoscope device 1c described above, the sheath 333 is inserted inside the object to be observed, for example, inside the ear or nose, the observation light is acquired via the optical scope 331, the first imaging element 921 and the second imaging element 922 receive the observation light and generate the imaging signal, and the imaging signal is imaged by the control device 31. In addition, it is possible to perform an incision or the like by causing the high-frequency current to flow using the resecto-electrode member 332 under the control of the control device 31 in the endoscope device 1c. At this time, it is possible to rotate the imaging adapter 342 with respect to the mounting member 341, and the user may change the position of the camera head 35 in the state of maintaining the position of the optical scope 331 using the grip handle 333a or the finger hooking handle 334a.

According to the fourth embodiment, it is possible to acquire the high-definition observation image, similarly to the above-described first embodiment, even in the endoscope, in particular, an endoscope for the ear or nose including an insertion unit with a small diameter. In addition, it is possible to obtain both the definition performance and the reduction in size even in a case in which an optical axis of the optical scope 331 and an optical axis of the camera head 35 are connected via the folded optical system as in the endoscope device described above.

In addition, the imaging adapter 342 is configured to rotate about the optical axis of the observation light, guided by the optical scope 331, with respect to the mounting member 341. Thus, it is possible to obtain the high-definition image regardless of rotation of the imaging adapter 342 according to the fourth embodiment.

### Fifth Embodiment

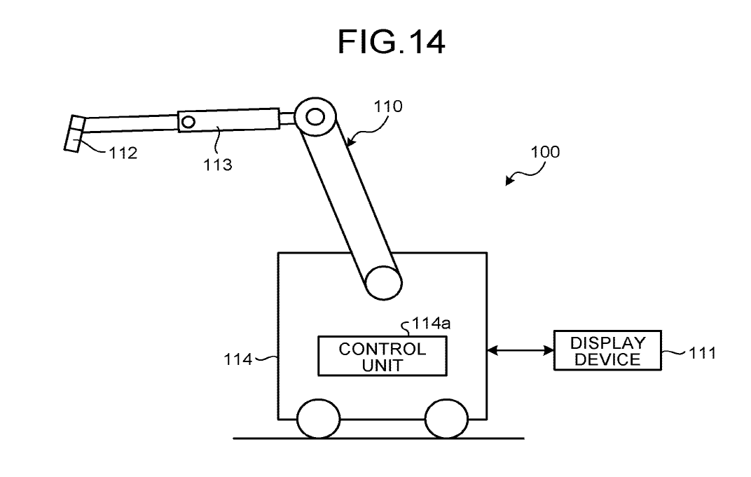

Next, a fifth embodiment of the present disclosure will be described. FIG. 14 is a diagram schematically illustrating the overall configuration of an operating microscope system which is an observation system for medical use provided with a medical imaging device according to a fifth embodiment. Although the description has been given by exemplifying the rigid endoscope in the above-described first and second embodiments, a description will be given in the fifth embodiment by exemplifying the operating microscope system (e.g., a medical image acquisition system) which has functions of magnifying and imaging a predetermined viewing area and displaying the captured image.

An operating microscope system 100 is provided with a microscope device 110 which is a medical imaging device that acquires an image to observe a subject by imaging, and a display device 111 which displays the image captured by the microscope device 110. Incidentally, the display device 111 may be configured to be integrated with the microscope device 110.

The microscope device 110 includes a microscope unit 112 which enlarges and images a microscopic part of the subject, a support unit 113 which is connected with a proximal end portion of the microscope unit 112 and includes an arm that supports the microscope unit 112 to be rotatable, and a base unit 114 which is rotatably hold a proximal end portion of the support unit 113 and is movable on a floor. The base unit 114 includes a control unit 114a which controls the operation of the operating microscope system 100. Incidentally, the base unit 114 may be configured to be fixed to a ceiling, a wall surface or the like and support the support unit 113 instead of being provided to be movable on the floor. In addition, the base unit 114 may be provided with a light source unit which generates illumination light such that the subjected is irradiated with illumination light from the microscope device 110.

The microscope unit 112 has, for example, a cylindrical shape, and includes the above-described imaging unit 92 therein. A switch, which receives input of an operation instruction of the microscope device 110, is provided in a side surface of the microscope unit 112. A cover glass (not illustrated) is provided in an opening surface of a lower end portion of the microscope unit 112 to protect the inside thereof.

A user such as a surgeon moves the microscope unit 112 or performs zooming while manipulating various types of switches in the state of gripping the microscope unit 112. Incidentally, it is preferable that a shape of the microscope unit 112 be a shape which extends to be elongated in an observation direction to allow the user to easily grip the unit and change a viewing direction. Thus, the shape of the microscope unit 112 may be a shape other than the cylindrical shape, and may have, for example, a polygonal column shape.

When the above-described imaging unit 92 is provided in the microscope unit 112 of the operating microscope system 100 having the above-described configuration, it is possible to generate the high-definition image while suppressing the increase in diameter of the microscope unit 112.

Although the embodiments of the present disclosure have been described so far, the present disclosure is not necessarily limited only by the embodiments described above. Although the description has been given, in the above-described embodiments, regarding a case in which the control device 5 performs the signal processing or the like, such processing may be performed by the camera head 9.

In addition, the description has been given by exemplifying the camera head (the camera head 9) as the rigid endoscope, the flexible endoscope, and the microscope device (e.g., the microscope device 110 of the operating microscope system 100) in the above-described embodiments. The definition of the imaging element, which is generally used in those devices, is set such that the flexible endoscope has the definition of SD (approximately, 720 in the row direction and 480 in the column direction), and the camera head and the microscope device have the definition of HD (approximately, 1920 in the row direction and 1080 in the column direction). When the imaging unit according to the embodiments is applied, it is possible to secure the high-quality observation image with the small and light device, and further, to enhance the definition of each device to be about doubled (e.g., SD to HD, and HD to 4K) by arranging the pixels of the two imaging elements to be relatively shifted.

As above, the medical imaging device, the medical image acquisition system, and the endoscope device according to the present disclosure are advantageous when acquiring the high-definition observation image while suppressing the increase in size of the device.

According to the present disclosure, it is possible to suppress the increase in size and weight of the device, and to acquire the high-quality observation image.

It should be understood by those skilled in the art that various modifications, combinations, sub-combinations and alterations may occur depending on design requirements and other factors insofar as they are within the scope of the appended claims or the equivalents thereof.

What is claimed is:

1. A medical imaging device comprising:
a spectroscopic unit configured to spectrally separate light from an outside into first light of a first color as a specific single color, and second light including specific second colors that are different from the first color;
a first imaging element configured to include first pixels, the first pixels receiving the first light spectrally separated by the spectroscopic unit and converting the first light into an electrical signal; and
a second imaging element configured to include:
second pixels arranged with same array and interval as the first pixels of the first imaging element; and
a color filter including filters each transmitting one of the second colors, the filters being arranged in accordance with the arrangement of the second pixels,
the second imaging element being configured to receive the second light transmitted through the color filter and to convert the second light into an electrical signal,
wherein the first and second imaging elements are arranged such that a pixel array of the second imaging element is shifted, with respect to a pixel array of the first imaging element, by a ½ pixel in at least one of two directions along an array direction when optical axes of the first and second light spectrally separated by the spectroscopic unit are set to match each other.

2. The medical imaging device according to claim 1, wherein
the first color is green,
the second colors are red and blue, and
the color filter includes the filters transmitting light of the red and light of the blue, respectively.

3. The medical imaging device according to claim 1, wherein the spectroscopic unit performs the spectral separation by reflecting one of the incident first and second light only once, and transmitting the other light.

4. The medical imaging device according to claim 3, wherein the spectroscopic unit is a prism formed by bonding two optical members with a dichroic film interposed therebetween, the dichroic film reflecting the one light and transmitting the other light.

5. The medical imaging device according to claim 3, wherein the spectroscopic unit is a plate-shaped member provided with a dichroic film on a surface thereof and having optical transparency, the dichroic film reflecting the one light and transmitting the other light.

6. The medical imaging device according to claim 1, further comprising
a gain adjustment unit configured to perform gain adjustment processing on each of signals generated by the first and second imaging elements.

7. The medical imaging device according to claim 1, wherein only the first and the second imaging elements are provided as imaging elements to image a subject and generate an imaging signal.

8. A medical image acquisition system comprising:
a medical imaging device including:
a spectroscopic unit configured to spectrally separate light from an outside into first light of a first color as a specific single color, and second light including specific second colors that are different from the first color;
a first imaging element configured to include first pixels, the first pixels receiving the first light spectrally separated by the spectroscopic unit and converting the first light into an electrical signal; and
a second imaging element configured to include:
second pixels arranged with same array and interval as the first pixels of the first imaging element; and
a color filter including filters each transmitting one of the second colors, the filters being arranged in accordance with the arrangement of the second pixels,
the second imaging element being configured to receive the second light transmitted through the color filter and to convert the second light into an electrical signal,
wherein the first and second imaging elements are arranged such that a pixel array of the second imaging element is shifted, with respect to a pixel array of the first imaging element, by a ½ pixel in at least one of two directions along an array direction when optical axes of the first and second light spectrally separated by the spectroscopic unit are set to match each other; and
an image processing device electrically connected with the medical imaging device and configured to receive the electrical signals, to process the received electrical signals and to generate an image signal according to the electrical signals.

9. An endoscope device comprising
a medical imaging device including:
a spectroscopic unit configured to spectrally separate light from an outside into first light of a first color as a specific single color, and second light including specific second colors that are different from the first color;
a first imaging element configured to include first pixels, the first pixels receiving the first light spectrally separated by the spectroscopic unit and converting the first light into an electrical signal; and
a second imaging element configured to include:
second pixels arranged with same array and interval as the first pixels of the first imaging element; and
a color filter including filters each transmitting one of the second colors, the filters being arranged in accordance with the arrangement of the second pixels,
the second imaging element being configured to receive the second light transmitted through the color filter and to convert the second light into an electrical signal,
wherein the first and second imaging elements are arranged such that a pixel array of the second imaging element is shifted, with respect to a pixel array of the first imaging element, by a ½ pixel in at least one of two directions along an array direction when optical axes of the first and second light spectrally separated by the spectroscopic unit are set to match each other.
